(19) United States
(12) Patent Application Publication
(10) Pub. No.: US 2022/0193180 A1
(43) Pub. Date: Jun. 23, 2022

(54) AGING PROGRESSION SUPPRESSING AGENT, AND FOOD OR BEVERAGE PRODUCT COMPRISING SAME

(71) Applicant: NITTA GELATIN INC., Osaka-shi, Osaka (JP)

(72) Inventor: Seiko KOIZUMI, Yao-shi, Osaka (JP)

(73) Assignee: NITTA GELATIN INC., Osaka-shi, Osaka (JP)

(21) Appl. No.: 17/606,124

(22) PCT Filed: Jul. 13, 2020
(86) PCT No.: PCT/JP2020/027261

§ 371 Date: Oct. 25, 2021

(30) Foreign Application Priority Data
Jul. 25, 2019 (JP) 2019-137126

(51) Int. Cl. A61K 38/06; A61K 38/05; A61P 17/18

(52) U.S. Cl. CPC A61K 38/06; A61P 17/18; A61K 38/05

(57) ABSTRACT

An aging progression suppressing agent comprises both or one of the peptides of Gly-Pro and Glu-Hyp-Gly, a salt thereof, or a chemically modified product thereof.

### TECHNICAL FIELD

The present invention relates to an aging progression suppressing agent, and a food or beverage product containing the same.

### BACKGROUND ART

One of the causes of aging may be oxidative stress given to various cells by active oxygen species, peroxides and the like. For example, Non Patent Literature 1 described below reports that graying (hereinafter, also referred to as “depigmentation”) of the hair of head progresses due to accumulation of the active oxygen species or peroxides in cells forming the hair follicle. Further, Non Patent Literatures 2 and 3 described below report that hair loss and depigmentation in the hair of head with aging is promoted by a decrease in type 17 collagen. Japanese Patent Laying-Open No. 2009-161509 (Patent Literature 1) discloses that the type 17 collagen has a function of suppressing hair loss and depigmentation in the hair of head.

### CITATION LIST

### Patent Literature

PTL 1: Japanese Patent Laying-Open No. 2009-161509

### Non Patent Literature

NPL 1: J M Wood et al., FASEB J, 2009, Vol 23, No. 7, pp. 2065-2075
NPL 2: Matsumura H et al., Science, 2016, Vol 351, pp. 575, add4395-1,2
NPL 3: Tanimura S et al., Cell Stem Cell, 2011, Vol 8, pp. 177-187

### SUMMARY OF INVENTION

### Technical Problem

On the other hand, collagen peptide mixtures obtained by performing hydrolysis on collagen or gelatin using a known proteolytic enzyme are known. The collagen peptide mixtures have been reported to have various physiological activities in the joint, the bone, the cartilage, the skin and the like within living organisms. However, it has not been heretofore reported that the collagen peptide mixtures have a suppressive action on hair loss and depigmentation in the hair of head. Glutathione is known as a peptide exhibiting a so-called antioxidant action of removing active oxygen species and peroxides from living organisms, and it has not been reported that the collagen peptide mixture is involved in synthesis of the glutathione. Thus, studies have been extensively conducted for exploring an aging progression suppressive action, specifically the suppressive action on hair loss and depigmentation in the hair of head, the glutathione synthesis promoting action, and the like, as new physiological activity of collagen peptide mixtures and collagen-derived peptides contained in the collagen peptide mixtures.

In view of the above-described circumstances, an object of the present invention is to provide an aging progression suppressing agent which comprises a peptide or the like exhibiting at least one of a promoting action on type 17 collagen gene expression and a promoting action on glutathione synthetase gene expression, and is thus capable of producing a suppressive effect on hair loss and depigmentation in the hair of head, or an antioxidant action enhancing effect; and a food or beverage product comprising the aging progression suppressing agent.

### Solution to Problem

In exploration of new physiological activity of a collagen peptide mixture, the present inventors have found that a predetermined peptide contained in a collagen peptide mixture exhibits at least one of a promoting action on type 17 collagen gene expression and a promoting action on glutathione synthetase gene expression. On the basis of the finding, an aging progression suppressing agent containing the peptide, thereby providing a suppressive effect on hair loss and depigmentation in the hair of head, or an antioxidant action enhancing effect has been attained, leading to completion of the present invention.

Specifically, the present invention is as follows.

The aging progression suppressing agent according to the present invention comprises both or one of the peptides of Gly-Pro and Glu-Hyp-Gly, a salt thereof, or a chemically modified product thereof.

Preferably, the peptides are derived from collagen.

Preferably, the aging progression suppressing agent is a collagen peptide mixture comprising any of the peptides.

Preferably, the collagen peptide mixture has a weight average molecular weight of 100 Da or more and 5,000 Da or less.

Preferably, the aging progression suppressing agent is a promoter of type 17 collagen gene expression or a promoter of glutathione synthetase gene expression.

The food or beverage product according to the present invention comprises the aging progression suppressing agent.

### Advantageous Effects of Invention

According to the present invention, it is possible to provide an aging progression suppressing agent capable of producing a suppressive effect on hair loss and depigmentation in the hair of head, or an antioxidant action enhancing effect; and a food or beverage product comprising the aging progression suppressing agent.

### DESCRIPTION OF EMBODIMENTS

Hereinafter, embodiments of the present invention will be described in more detail. As used herein, the notation in the form of “A to B” means the upper limit and the lower limit of a range (i.e. A or more and B or less), and when a unit is not described for A, and a unit is described only for B, the unit for A is identical to the unit for B.

[Aging Progression Suppressing Agent]

The aging progression suppressing agent according to the present invention comprises both or one of the peptides of Gly-Pro and Glu-Hyp-Gly, a salt thereof, or a chemically modified product thereof. The aging progression suppressing agent having such a characteristic can exhibit a promoting action on type 17 collagen gene expression or a promoting action on glutathione synthetase gene expression, and therefore it is possible to obtain a suppressive effect on hair loss and depigmentation in the hair of head or an antioxidant action enhancing effect.

[Both or One of Peptides of Gly-Pro and Glu-Hyp-Gly, Salt Thereof, or Chemically Modified Product Thereof]

As described above, the aging progression suppressing agent comprises both or one of the peptides of Gly-Pro and Glu-Hyp-Gly, a salt thereof, or a chemically modified product thereof. In the present description, the “amino acid” forming the peptide is represented by a three-character abbreviation unless otherwise specified. Further, the “amino acid” means an L-type amino acid unless otherwise specified. Further, for the “peptide” in the present description, for example, “Gly-Pro” means a peptide (dipeptide) in which glycine and proline are arranged in this order from the N-terminal side toward the C-terminal side, and “Glu-Hyp-Gly” means a peptide (tripeptide) in which glutamic acid, hydroxyproline and glycine are arranged in this order from the N-terminal side toward the C-terminal side. The same applies to the descriptions of peptides other than “Gly-Pro” and “Glu-Hyp-Gly”.

Preferably, the aging progression suppressing agent comprises both the peptides of Gly-Pro and Glu-Hyp-Gly, a salt thereof, or a chemically modified product thereof. In this case, the aging progression suppressing agent can more markedly exhibit a promoting action on type 17 collagen gene expression or a promoting action on glutathione synthetase gene expression.

The term “salt” of the peptide is formed as, for example, an inorganic acid salt such as a hydrochloride, a sulfate or a phosphate, an organic acid salt such as a methanesulfonate salt, a benzenesulfonate salt, a succinate salt or an oxalate salt, an inorganic basic salt such as a sodium salt, a potassium salt or a calcium salt, an organic basic salt such as a triethylammonium salt, of the peptide.

The “chemically modified product” of the peptide means a compound in which a free functional group of an amino acid residue that is a constituent unit is chemically modified. Chemical modification can be performed on, for example, a hydroxyl group of hydroxyproline, an amino group of an amino acid on the N-terminal (amino terminal) side and a carboxyl group of an amino acid on the C-terminal (carboxyl terminal) side. For specific means and treatment conditions for chemical modification, known conventional chemical modification techniques targeting amino acids and peptides are applied. The chemically modified product of each of the amino acids and peptides, which is obtained by such chemical modification, can produce an enhancing effect on solubility under a mildly acidic to neutral condition, an enhancing effect on compatibility with other active ingredients, and the like.

For example, the tripeptide of Glu-Hyp-Gly can be subjected to O-acetylation as chemical modification of a hydroxyl group in hydroxyproline. The O-acetylation can be performed by applying acetic anhydride to the peptide in an aqueous solvent or a nonaqueous solvent. Esterification, amidation or the like can be performed as chemical modification of a carboxyl group in glycine. The esterification can be performed by suspending the peptide in methanol, and then causing dry hydrogen chloride gas to pass through the resulting suspension. The amidation can be performed by applying carbodiimide or the like to the peptide.

Methylation can be performed as chemical modification of a free amino group in the peptide. At least one of phosphorylation and sulfation can be performed as chemical modification of a free hydroxyl group in the peptide.

Preferably, the peptide is derived from collagen. Here, the collagen as a raw material can be obtained by performing known conventional defatting or decalcification treatment, extraction treatment or the like on, for example, the skin, the dermis, the bone, the cartilage, the tendon or the like of animals typically of a bovine, a pig, a sheep, a chicken or an ostrich, or the bone, the skin, the scale or the like of fish. Further, gelatin can be used as a raw material for the peptide. The gelatin can be obtained by treating the thus-obtained collagen through a known conventional method such as extraction with hot water. For the collagen and the gelatin, commercial products can be used as raw materials.

The peptide can be obtained by hydrolyzing the collagen and/or the gelatin with two or more of endo-type proteases and exo-type proteases in combination. By the hydrolysis, the peptide can be obtained as a collagen peptide mixture in which the peptide is present together with other collagen peptides. The collagen peptide mixture itself and a mixture obtained by partially purifying the collagen peptide mixture can be used as the aging progression suppressing agent according to the present invention. That is, the aging progression suppressing agent is preferably a collagen peptide mixture. Further, by further purifying the collagen peptide mixture, a purified product containing the peptide can be obtained with a high purity. When the peptide is derived from collagen, it is preferable to obtain the peptide by using a method in which collagen or gelatin is enzyme-treated in two stages as described below.

Further, the weight average molecular weight of the collagen peptide mixture is preferably 100 Da or more and 5,000 Da or less. The weight average molecular weight of the collagen peptide mixture is more preferably 120 Da or more and 3,500 Da or less, still more preferably 150 Da or more and 3,000 Da or less. When the weight average molecular weight of the collagen peptide mixture is within the above-described range, the aging progression suppressing agent can sufficiently produce a promoting action on type 17 collagen gene expression or a promoting action on glutathione synthetase gene expression. If the weight average molecular weight is more than 5,000 Da, the above-described effect of the aging progression suppressing agent may be insufficient.

The weight average molecular weight of the collagen peptide mixture can be determined by carrying out size exclusion chromatography (SEC) under the following measurement conditions.

Equipment: High-performance liquid chromatography (HPLC) (manufactured by TOSOH CORPORATION)
Column: TSKGel (registered trademark) G2000SW_{XL}
Column temperature: 40° C.
Colum size: 7.8 mm (I.D.)×30 cm, 5 μm
Eluant: 45 mass % acetonitrile (with 0.1 mass % trifluoroacetic acid)
Flow rate: 1.0 mL/min
Injection amount: 10 μL

### Detection: UV 214 nm

Molecular weight marker: The following five types are used

| | |
|---|---|
| Cytochrome C | Mw: 12,000 |
| Aprotinin | Mw: 6,500 |
| Bacitracin | Mw: 1,450 |
| Gly-Gly-Tyr-Arg | Mw: 451 |
| Gly-Gly-Gly | Mw: 189 |

Specifically, a sample comprising about 0.2 g of the collagen peptide mixture is added to about 100 ml of distilled water, the mixture is stirred, and then filtered with a 0.2 μm filter to prepare a sample of which weight average molecular weight is measured (measurement specimen). By subjecting the measurement specimen to the size exclusion chromatography, the weight average molecular weight of the collagen peptide mixture can be determined.

[Method for Producing Aging Progression Suppressing Agent]

The peptide contained in the aging progression suppressing agent can be obtained by known conventional methods. For example, the peptide can be obtained by purchasing commercially available amino acids. The peptide can also be obtained by using a method including hydrolyzing collagen or gelatin.

The peptides (both or one of Gly-Pro and Glu-Hyp-Gly) can be each obtained by a known conventional liquid-phase or solid-phase peptide synthesis method, or a method including hydrolyzing collagen or gelatin. From the viewpoint of efficiency, it is preferable to produce the peptide by using a chemical synthesis method using an amino acid as described below, or a method including enzymatically treating collagen or gelatin in two stages as described below. Further, the peptide can be produced by using a method including performing enzymatic treatment with only a secondary enzyme with a primary enzyme omitted, or a method including performing enzymatic treatment with a primary enzyme and a secondary enzyme simultaneously, instead of the method including enzymatically treating collagen or gelatin in two stages. Hereinafter, a method for producing, in particular, “Glu-Hyp-Gly”, among the peptides contained in the aging progression suppressing agent, will be described as an example of a method for producing a peptide contained in the aging progression suppressing agent.

<Chemical Synthesis Method>

The peptide can be obtained by using a common peptide synthesis method. As the peptide synthesis method, a solid-phase synthesis method and a liquid-phase synthesis method are known. As the solid-phase synthesis method, an Fmoc method and a Boc method are known. The peptide can be obtained by using either of the Fmoc method and the Boc method. As the solid-phase peptide synthesis method, a method for synthesizing a tripeptide represented by Glu-Hyp-Gly can be carried out as follows.

First, a bead of a polystyrene polymer gel having a diameter of about 0.1 mm and having a surface modified with amino groups is provided as a solid phase. Separately, diisopropylcarbodiimide is provided as a condensing agent. Next, the amino group of glycine, which is an amino group on the C-terminal (carboxyl terminal) side in the amino acid sequence, is protected with an Fmoc (fluorenyl-methoxy-carbonyl) group, the carboxyl group of the glycine is peptide-bound to the amino group as the solid phase through a dehydration reaction using the condensing agent. Further, the solid phase is washed with a solvent to remove the remaining condensing agent and amino acids, followed by removing the protecting group (deprotecting) of the amino group of glycine which is peptide-bound to the solid phase.

Subsequently, hydroxyproline in which an amino group is protected with an Fmoc group is provided, and the carboxyl group of the hydroxyproline is peptide-bound to the deprotected amino group of the glycine by using the condensing agent. Thereafter, in the same manner as described above, the amino group of the hydroxyproline is deprotected, glutamic acid protected with an Fmoc group is provided, and a reaction for peptide-binding the glutamic acid to the hydroxyproline is carried out to synthesize a tripeptide represented by Glu-Hyp-Gly as the solid phase. Finally, the tripeptide can be produced by deprotecting the amino group of the glutamic acid, and separating the tripeptide from the solid phase by immersion in trifluoroacetic acid under heating.

<Production Method Using Collagen and Gelatin>

Further, a method for enzymatically treating collagen or gelatin in two stages to produce a tripeptide represented by Glu-Hyp-Gly can be carried out as follows.

The term “enzymatically treating (collagen or gelatin) in two stages” means the following. That is, primary enzymatic treatment is performed by a known conventional method for breaking the peptide bond of collagen or gelatin, and secondary enzymatic treatment is then performed with an enzyme having aminopeptidase N activity, an enzyme having both aminopeptidase N activity and prolyl tripeptidyl aminopeptidase activity, or a combination of an enzyme having aminopeptidase N activity and an enzyme having prolyl tripeptidyl aminopeptidase activity. By performing the primary enzymatic treatment, a collagen peptide mixture precursor can be obtained. By further performing the secondary enzymatic treatment, a collagen peptide mixture comprising the Glu-Hyp-Gly can be obtained from the collagen peptide mixture precursor. The method for enzymatically treating collagen or gelatin in two stages will be described in more detail below.

(Primary Enzymatic Treatment)

The enzyme used in the primary enzymatic treatment should not be particularly limited as long as it is an enzyme capable of breaking peptide bonds of collagen or gelatin, and any proteolytic enzyme can be used. Specifically, examples of thereof include collagenase, thiol protease, serine protease, acidic protease, alkaline protease and metal protease. One selected from the group consisting of these enzymes may be used alone, or two or more thereof may be used in combination. As the thiol protease, chymopapain, papain, bromelain and ficin derived from plants, cathepsin and calcium dependent protease derived from animals, and the like can be used. As the serine protease, trypsin, cathepsin D and the like can be used. As the acidic protease, pepsin, chymotrypsin and the like can be used. Considering that the aging progression suppressing agent according to the present invention is used for medicaments, specified health food and the like, it is preferable that as the enzymes used in the primary enzymatic treatment, those other than enzymes derived from pathogenic microorganisms be used.

The amount of enzymes in the primary enzymatic treatment is, for example, preferably 0.1 to 5 parts by mass of the above-described enzymes based on 100 parts by mass of collagen or gelatin. Preferably, the treatment temperature and the treatment time in the primary enzymatic treatment are 30 to 65° C. and 10 minutes to 72 hours, respectively. The weight average molecular weight of the collagen peptide mixture precursor obtained through the primary enzymatic treatment is preferably 500 to 20,000 Da, more preferably 500 to 10,000 Da, still more preferably 500 to 8,000 Da. It can be said that when the weight average molecular weight is within the above-described range, a peptide having an appropriate molecular weight is adequately generated. If necessary, the enzyme can be deactivated after the primary enzymatic treatment. In this case, the deactivation temperature is, for example, preferably 70 to 100° C. The weight average molecular weight of the collagen peptide mixture precursor can be determined by the method using SEC.

(Secondary Enzymatic Treatment)

Examples of the enzyme used in the secondary enzymatic treatment include enzymes having aminopeptidase N activity, enzymes having both aminopeptidase N activity and prolyl tripeptidyl aminopeptidase activity, and combinations of an enzyme having aminopeptidase N activity and prolyl tripeptidyl aminopeptidase activity. The term “enzyme having aminopeptidase N activity” as used herein is a peptidase having a function of releasing an amino acid from the N-terminal side of the peptide chain, where the enzyme acts when an amino acid other than proline or hydroxyproline exists at the second position from the N-terminal side. The term “enzyme having prolyl tripeptidyl aminopeptidase activity” as used herein is a peptidase which releases only three amino acid residues on the N-terminal side from a peptide having proline or hydroxyproline at the third position from the N-terminal side. Considering that the aging progression suppressing agent according to the present invention is used for medicaments, specified health food and the like, it is preferable that as the enzymes used in the secondary enzymatic treatment, those other than enzymes derived from pathogenic microorganisms be used.

Examples of the enzyme having aminopeptidase N activity include aminopeptidase N (EC 3.4.11.2.; T. Yoshimoto et al., Agric. Biol. Chem., 52: 217-225 (1988)), and enzymes having aminopeptidase N activity derived from Aspergillus. Examples of the enzyme having prolyl tripeptidyl aminopeptidase activity include prolyl tripeptidyl aminopeptidase (EC 3.4.14.; A. Banbula et al., J. Biol. Chem., 274: 9246-9252 (1999)).

By performing the secondary enzymatic treatment, a collagen peptide mixture containing a peptide which has not been contained in the collagen peptide mixture precursor can be obtained. Specifically, a collagen peptide mixture containing the Glu-Hyp-Gly can be obtained.

The amount of enzymes in the secondary enzymatic treatment is, for example, preferably 0.01 to 5 parts by mass of the above-described enzymes based on 100 parts by mass of the collagen peptide mixture precursor. Preferably, the treatment temperature and the treatment time in the secondary enzymatic treatment are 30 to 65° C. and 10 minutes to 72 hours, respectively. The weight average molecular weight of the collagen peptide mixture obtained through the secondary enzymatic treatment is preferably 100 to 5,000 Da, more preferably 120 to 3,500 Da, still more preferably 150 to 3,000 Da. The weight average molecular weight of the collagen peptide mixture can also be determined by the method using SEC described above.

The secondary enzymatic treatment is performed mainly for the purpose of generating the tripeptide of Glu-Hyp-Gly. Thus, it is preferable to adjust the amount of enzymes, the treatment temperature, the treatment time and the pH in the secondary enzymatic treatment so that the peptide contained in the collagen peptide mixture precursor is not excessively hydrolyzed. Accordingly, the weight average molecular weight of the collagen peptide mixture is preferably within the above-described range. It is necessary to deactivate the enzyme after the secondary enzymatic treatment. In this case, the deactivation temperature is, for example, preferably 70 to 100° C. Further, it is preferable to perform sterilization treatment at 120° C. for several seconds or more. In addition, the collagen peptide mixture can be subjected to spray drying by applying heat at 200° C. or higher.

In the secondary enzymatic treatment, not only the enzymes having aminopeptidase N activity and enzymes having prolyl tripeptidyl aminopeptidase activity, but also enzymes having different activities can be used, and two or more enzymes each having different activities can be used in combination. Consequently, by-products can be digested and removed. Preferably, the enzymes used in this case are appropriately selected, depending on the type of collagen used as a raw material, and the type of enzyme used in the primary enzymatic treatment. Examples of the different activities include dipeptidase activity such as prolidase activity and hydroxyprolidase activity. Consequently, by-products such as dipeptides can be digested and removed.

Further, the aminopeptidase N activity is basically activity causing the release of amino acids on the N-terminal side one by one. Thus, when the secondary enzymatic treatment is performed only with an enzyme having aminopeptidase N activity in the case where the collagen peptide mixture precursor obtained through the primary enzymatic treatment contains a peptide having an extremely large molecular weight, the duration for the secondary enzymatic treatment markedly increases. For coping with such a case, for example, prolyl oligopeptidase which is an endopeptidase having activity causing hydrolysis of proline on the carboxyl group side (prolidase activity) can be used in the secondary enzymatic treatment. Consequently, the secondary enzymatic treatment can be efficiently performed.

In the method including enzyme-treating collagen or gelatin in two stages, the primary enzymatic treatment enables generation of a peptide having a relatively large molecular weight. This peptide can have an amino acid sequence represented by, for example, [X₁-Gly-X₂-Glu-Hyp-Gly] (X₁ and X₂≠Hyp). In the subsequent secondary enzymatic treatment, an enzyme having aminopeptidase N activity acts on the peptide represented by [X₁-Gly-X₂-Glu-Hyp-Gly], so that X₁ at the N-terminal is released to obtain a peptide having an amino acid sequence represented by [Gly-X₂-Glu-Hyp-Gly].

Next, an enzyme having aminopeptidase N activity acts twice on the peptide represented by [Gly-X₂-Glu-Hyp-Gly], so that glycine and X₂ are released to obtain a peptide represented by [Glu-Hyp-Gly].

(Purification of Collagen Peptide Mixture) By performing enzymatic treatment in two stages as described above, a collagen peptide mixture containing Glu-Hyp-Gly can be produced. Since the collagen peptide mixture contains peptides other than the tripeptide represented by Glu-Hyp-Gly, it is preferable to purify the collagen peptide mixture if necessary. As a purification method in this case, a known conventional method can be used, and examples thereof include ultrafiltration, and various types of liquid chromatography such as size exclusion chromatography, ion-exchange chromatography, reversed phase chromatography and affinity chromatography.

Specifically, the collagen peptide mixture can be purified in accordance with the following procedure. That is, about 2 g/10 ml of the collagen peptide mixture is loaded into an ion-exchange column (e.g. “TOYOPEARL” (registered trademark) DEAE-650″ (trade name) manufactured by TOSOH CORPORATION), and a first void volume fraction eluted with distilled water is then collected. Subsequently, the first void volume fraction is loaded into a column having an ion-exchange group opposite to that of the above ion-exchange column (e.g. “TOYOPEARL” (registered trademark) SP-650 manufactured by TOSOH CORPORATION), and a second void volume fraction eluted with distilled water is then collected.

Next, the second void volume fraction is loaded into a gel filtration column (e.g. “SEPHADEX LH-20” (trade name) manufactured by GE Healthcare Japan Corporation), and eluted with a 30 mass % methanol aqueous solution to collect a fraction containing the tripeptide of Glu-Hyp-Gly. Finally, using a high-performance liquid chromatography (HPLC) with a reversed-phase column (e.g. “μBondasphere 5μ C18 300 Å Column” (trade name) manufactured by Waters Corporation), the fraction is fractionated in accordance with a linear concentration gradient of a 32 mass % or less acetonitrile aqueous solution containing 0.1 mass % trifluoroacetic acid. In this way, Glu-Hyp-Gly can be obtained with a high purity.

[Promoter of Type 17 Collagen Gene Expression or Promoter of Glutathione Synthetase Gene Expression]

The aging progression suppressing agent according to the present invention is preferably a promoter of type 17 collagen gene expression or a promoter of glutathione synthetase gene expression. The aging progression suppressing agent comprises both or one of the peptides of Gly-Pro and Glu-Hyp-Gly, a salt thereof, or a chemically modified product thereof as described above. This enables exhibition of a promoting action on type 17 collagen gene expression. Thus, the aging progression suppressing agent promotes type 17 collagen gene expression as a promoter of type 17 collagen gene expression, and therefore can suppress hair loss and depigmentation in the hair of head. The promoter of type 17 collagen gene expression promotes type 17 collagen gene expression, and therefore can be expected to exhibit a suppressive effect on progression of age-related hair thinning, hair loss and graying, a skin beautification promoting effect, and the like.

Further, the aging progression suppressing agent comprises the peptide, a salt thereof, or a chemically modified product thereof, and therefore can exhibit a promoting action on glutathione synthetase gene expression. Thus, the aging progression suppressing agent promotes glutathione synthetase gene expression as a promoter of glutathione synthetase gene expression, and therefore enables removal of active oxygen species, peroxides and the like from living organisms. The promoter of glutathione synthetase gene expression enables removal of active oxygen species, peroxides and the like from living organisms, and therefore can also be expected to exhibit effects such as skin whitening based on suppression of pigment deposition due to inflammation, skin beautification based on suppression of eczema, promotion of healing of corneal injury, improvement in hepatic function, and improvement in Parkinson's disease.

The aging progression suppressing agent can be orally or parenterally administered in various forms. For these forms, the aging progression suppressing agent can take dosage forms such as tablets, granules, capsules, powders, liquids, suspension preparations and emulsion preparations when orally administered. Further, the aging progression suppressing agent in any of the above-described dosage forms can also be mixed with a food or beverage product. The aging progression suppressing agent comprises any of the peptides, which are rapidly absorbed in the intestinal tract, and therefore can be taken via oral administration.

When parenterally administered, the aging progression suppressing agent can be in the dosage forms such as external preparations such as ointments, creams and lotions, and transdermal preparations. Further, the aging progression suppressing agent can be in the forms of solutions or coatings to be rubbed directly into the head skin. When the aging progression suppressing agent is used as a coating, the concentration of the peptide or the like contained in the coating is preferably 0.001 to 5 mass %.

The dose of the aging progression suppressing agent varies depending on the age, the sex, the body weight and the sensitivity difference of a subject, the administration method, the administration interval, the type of preparation and the like. When the aging progression suppressing agent is orally administered, the dose per adult is, for example, preferably 0.0001 to 2,500 mg/kg, more preferably 0.0001 to 500 mg/kg. When the dosage form of the aging progression suppressing agent is, for example, a tablet, the tablet may contain the aging progression suppressing agent in an amount of 0.001 to 80 mass % per tablet, and when the dosage form of the aging progression suppressing agent is, for example, a powder, the powder may contain the aging progression suppressing agent in an amount of 0.001 to 100 mass %. When the aging progression suppressing agent is parenterally administered or administered by a preparation in another form, the dose can be appropriately determined by reference to a dose in oral administration. The aging progression suppressing agent can be administered daily once or in several divided doses, or administered once every day or every several days.

The aging progression suppressing agent may appropriately contain other active ingredients, a preparation carriers and the like as long as the effects of the present invention are not adversely affected. Examples of other active ingredients include inulin, caffeic acid, quinic acid, derivatives thereof, extracts from marjoram, crude drugs such as Kinfukan, milkwort (polygalae radix), Hakubiso and Desmos chinensis Lour, royal jerry, extracts from echinacea, extracts from acai, and extracts from Cupuacu. Further, examples of pharmaceutically acceptable carriers used in formulation into pharmaceutical preparations include diluents, binding agents (syrup, gum arabic, gelatin, sorbitol, tragacanth and polyvinylpyrrolidone), excipients (lactose, sucrose, cornstarch, potassium phosphate, sorbitol and glycine), lubricants (magnesium stearate, talc, polyethylene glycol and silica), disintegrants (potato starch) and wetting agents (sodium lauryl sulfate).

[Use Invention]

The aging progression suppressing agent according to the present invention comprises both or one of the peptides of Gly-Pro and Glu-Hyp-Gly, a salt thereof, or a chemically modified product thereof as described above. The aging progression suppressing agent can exhibit at least one of a promoting action on type 17 collagen gene expression or a promoting action on glutathione synthetase gene expression as an attribute of the peptide. In other words, the present invention is a peptide, a salt thereof or a chemically modified product thereof which has newly found a use for suppressing progression of aging on the basis of the attribute.

[Food or Beverage Product]

The food or beverage product according to the present invention comprises the aging progression suppressing agent. For example, the peptide preferably contained in the aging progression suppressing agent is rapidly absorbed in the intestinal tract as described above, and therefore can be taken via oral administration. Thus, the aging progression suppressing agent of the present invention can be taken as a food or beverage product in which the aging progression suppressing agent is mixed with food or a beverage. Further, the aging progression suppressing agent according to the present invention can be used as specified health food or food with functional claims. The concentration of the aging progression suppressing agent contained in the food or beverage product is preferably 0.001 to 100 mass %.

### EXAMPLES

Hereinafter, the present invention will be described in more detail by way of Example, which should not be construed as limiting the present invention.

### Example 1

### [Preparation of Sample]

### <Preparation of Peptide and Collagen Peptide Mixture>

The peptides and collagen peptide mixtures shown in Tables 1 to 4 below were provided by production using the above-described methods or purchase from the manufacturers described later. The peptides and collagen peptide mixtures serve as specimens for determining whether or not they have an effect on the messenger RNA level (mRNA level) of the type 17 collagen gene and the mRNA level of the glutathione synthetase gene in the epidermal cells described later.

Here, for the peptides shown in Tables 1 and 2, abbreviations in which amino acids forming the peptides are each represented by one character are used. In Table 1, “EO” represents a dipeptide of glutamic acid-hydroxyproline (manufactured by PH Japan Co., Ltd.). “GP” represents a dipeptide of glycine-proline (trade name: “G-3015”, manufactured by BACHEM Co.). “EOG” represents a tripeptide of glutamic acid-hydroxyproline-glycine (manufactured by PH Japan Co., Ltd.).

Further, the collagen peptide mixture A (trade name: “COLLAPEP PU”, manufactured by Nitta Gelatin Inc., weight average molecular weight (Mw): about 630 Da) shown in Table 3 was found to contain “EOG” and “GP” in the following amounts in quantitative analysis performed by LC-MS/MS under the conditions described later. Glu-Hyp-Gly: 4 ppm, Gly-Pro: 2,379 ppm, total: 2,383 ppm.

Next, the collagen peptide mixture B (trade name: “TYPE-S”, manufactured by Nitta Gelatin Inc., weight average molecular weight (Mw): about 750 Da) shown in Table 4 was found to contain “EOG” and “GP” in the following amounts in quantitative analysis performed by LC-MS/MS under the conditions described later. Glu-Hyp-Gly: 9 ppm, Gly-Pro: 1,159 ppm, total: 1,168 ppm.

The collagen peptide mixture C shown in Table 4, which is a collagen peptide mixture that is being developed by Nitta Gelatin Inc. (weight average molecular weight (Mw): about 450 Da), was found to contain “EOG” and “GP” in the following amounts in quantitative analysis performed by LC-MS/MS under the conditions described later. Glu-Hyp-Gly: 24 ppm, Gly-Pro: 26,387 ppm, total: 26,411 ppm.

The quantitative analysis by LC-MS/MS was performed under the following conditions.

HPLC apparatus: “ACQUITY UPLC H-Class Bio”, manufactured by Waters Corporation)
Column: “Hypersil GOLD PFP 2.1×150 mm, 5 μM (manufactured by Thermo Fisher Scientific. Inc.)
Column temperature: 40° C. (linear gradient)
Mobile phase: (A) aqueous solution containing 0.2% formic acid and 2 mM ammonium acetate

(B) 100% methanol
(Gradient Setting)

| Time (min) | Flow rate | Mobile phase (mass %) |
|---|---|---|
| Initial | 200 | 98 |
| 3.50 | 200 | 98 |
| 3.51 | 400 | 5 |
| 7.00 | 400 | 5 |
| 7.10 | 200 | 98 |
| 17.00 | 200 | 98 |

Injection amount: 0.5 μl

MS/MS Apparatus: “Xevo TQ-XS” manufactured by Waters Corporation Ionization method: Positive ESI

### Capillary (kV): 1

Desolvation temperature (° C.): 500
Source temperature (° C.): 150
MRM conditions:

| Peptide (abbreviation) | precursor ion (m/z) | product ion (m/z) |
|---|---|---|
| Gly-Pro (GP) | 173 | 116 |
| Glu-Hyp-Gly (EOG) | 318 | 225 |

<Preparation of Epidermal Cells>

First, normal human epidermal keratinocytes NHEK (NB) (manufactured by KURABO INDUSTRIES LTD.) were obtained as epidermal cells. The cells were seeded in a necessary number of commercially available dishes of ϕ60 mm at 1.25×10⁴ cells per dish (5 mL of a cell dispersion liquid having a concentration of 0.25×10⁴ cells/mL), and cultured in a serum-free medium (trade name: “HuMedia KG-2”, manufactured by KURABO INDUSTRIES LTD.) for 2 days. Then, the cells were confirmed to be subconfluent in the dishes, and the medium in the dishes was then replaced by a basal medium (trade name: “HuMedia KB-2”, manufactured by KURABO INDUSTRIES LTD.). In this way, epidermal cells for evaluating the mRNA level of the type 17 collagen gene and the mRNA level of the glutathione synthetase gene were prepared.

<Gene Expression Test>

To the basal medium in the dishes, the peptide or the collagen peptide mixture was added to the concentrations shown in Tables 1 to 4, and the cells were cultured at 37° C. in an atmosphere at a carbon dioxide concentration of 5 vol % for 72 hours to prepare samples to be subjected to a gene expression test. In addition, a control sample obtained by adding only ion-exchanged water to the basal medium in the dish (hereinafter, also referred to as “Blank”) was prepared. This control sample was also cultured at 37° C. in an atmosphere at a carbon dioxide concentration of 5 vol % for 72 hours.

Next, by using a RNA extraction kit (trade name: “TRIzol (registered trademark) Reagent, manufactured by Life Technologies Japan Ltd.) in accordance with the protocol accompanying the kit, total RNA was extracted from the epidermal cells in the dish to obtain an extract containing total RNA for each sample. Subsequently, by using a cDNA preparation kit (trade name (product number): “High Capacity RNA-to-cDNA Kit (4387406)”, manufactured by Life Technologies Japan Ltd.) in accordance with the protocol accompanying the kit, reverse transcription was performed on RNA in the extract to obtain cDNA from the RNA in the extract. Further, real-time (RT)-PCR was performed on the cDNA by a DNA amplifying apparatus (trade name: “Step One Plus (TM) Real-Time PCR System”, manufactured by Applied Biosystems Inc.).

In the RT-PCR, the mRNA levels of type 17 collagen (manufactured by Life Technologies Japan Ltd., primer: Hs009900361_ml) as a target gene and glutathione synthetase (GSS, manufactured by Life Technologies Japan Ltd., primer: Hs01547656_ml) were measured. As an internal standard (correction gene), GAPDH was selected. For calculation of the mRNA level, a calibrated curve method was used. As the primer and the probe for the RT-PCR, those accompanying a reagent kit (trade name: “TaqMan (registered trademark) Gene Expression Assays, manufactured by Applied Biosystems Inc.) were used.

Data obtained from the RT-PCR was analyzed as follows. First, in the samples and the control sample, the mRNA levels (gene expression levels) of the two target genes (type 17 collagen and glutathione synthetase) were each calculated. Next, the mRNA levels of the two target genes were corrected with the mRNA level of GAPDH as a correction gene to obtain correction values in the samples and the control sample. Specifically, values obtained by dividing the mRNA levels of the two target genes by the mRNA level of GAPDH (relative values) were each determined.

Then, the ratio of the correction value obtained in each sample to the correction value in the control sample, which was defined as 100, was determined (gene expression increase rate (%)). The effects of addition of the peptide and the collagen peptide mixture on the mRNA level of the type 17 collagen gene and the mRNA level of the glutathione synthetase gene in the epidermal cells (whether the promoting action on gene expression was exhibited or not) were evaluated.

Further, the gene expression increase rate (%) was subjected to statistical processing to evaluate significance of the promoting action on gene expression of the type 17 collagen gene and the glutathione synthetase gene in each sample. For the evaluation of significance, statistical processing was performed using software (“Excel (Ver 2016)” (trade name) manufactured by Social Survey Research Information Co., Ltd.), Smirnov-Grubbs (two-sided test) was conducted, and the significance level (P value) was set to 0.01 as a threshold. Thereafter, the Student's t-test (t-test) was conducted to evaluate significance. Tables 1 to 4 show the results. In Tables 1 to 4, samples with “++” were determined to have a significance in the promoting action on expression of the gene. In samples with “+”, the gene expression increase rate (%) exceeded 100. Samples with “−” were determined to have no significance in the promoting action on expression of the gene.

Here, Table 1 shows the gene expression increase rates of type 17 collagen gene when the peptides of “EO”, “GP” and “EOG” were each added to the epidermal cells. Table 2 shows the gene expression increase rates of the glutathione synthetase gene when the peptides of “GP” and “EOG” were each added to the epidermal cells. Table 3 shows the gene expression increase rates of type 17 collagen gene when the “collagen peptide mixture A” was added to the epidermal cells. Table 4 shows the amounts of increase in gene expression increase rate of the glutathione synthetase gene when the “collagen peptide mixture B” and the “collagen peptide mixture C” were each added to the epidermal cells.

TABLE 1

| | | Gene | Ratio to | |
|---|---|---|---|---|
| Peptide | Content (final | expression | control | |
| (sample) | concentration) | increase rate | t-test | Assessment |
| Blank | 0 mM | 100 ± 0.87 | — | − |
| EO | 1 mM | 100 ± 6.46 | 0.934 | − |
| GP | 1 mM | 201 ± 8.42 | 0.00007 | ++ |
| EOG | 1 mM | 144 ± 17.01 | 0.024 | + |

TABLE 2

| | Content | Gene | Ratio to | |
|---|---|---|---|---|
| Peptide | (final | expression | control | |
| (sample) | concentration) | increase rate | t-test | Assessment |
| Blank | 0 mM | 100 ± 11 | — | − |
| GP | 1 mM | 106 ± 2 | 0.292 | + |
| EOG | 1 mM | 131 ± 10 | 0.024 | + |

TABLE 3

| | Content | Gene | Ratio to | |
|---|---|---|---|---|
| Collagen peptide | (mass %) (final | expression | control | |
| mixture (sample) | concentration) | increase rate | t-test | Assessment |
| Blank | — | 100 ± 5.08 | — | − |
| Collagen peptide | 0.05% | 132 ± 5.08 | 0.03 | + |
| Mixture A | | | | |

TABLE 4

| | Content | Gene | Ratio to | |
|---|---|---|---|---|
| Collagen peptide | (mass %) (final | expression | control | |
| mixture (sample) | concentration) | increase rate | t-test | Assessment |
| Blank | — | 100 ± 11 | — | − |
| Collagen peptide | 0.5% | 189 ± 10 | 0.001 | ++ |
| Mixture B | | | | |
| Collagen peptide | 0.5% | 202 ± 5 | 0.0003 | ++ |
| Mixture C | | | | |

[Discussions]

From Tables 1 to 4, it is understood that a sample comprising both or one of the peptides of Gly-Pro (GP) and Glu-Hyp-Gly (BOG) has at least one of a promoting action on type 17 collagen gene expression and a promoting action on glutathione synthetase gene expression. The collagen peptide mixtures A to C containing these peptides also had at least one of a promoting action on type 17 collagen gene expression and a promoting action on glutathione synthetase gene expression. On the other hand, a sample comprising the peptide of Glu-Hyp (EO) did not exhibit an evident promoting action on type 17 collagen gene expression. This indicates that as aging progression suppressing agents, the peptides of Gly-Pro and Glu-Hyp-Gly and collagen peptide mixtures comprising these peptides had an effect of suppressing hair loss and depigmentation in the hair of head by promoting type 17 collagen gene expression. Further, it is indicated that as aging progression suppressing agents, the above-described peptides and collagen peptide mixtures comprising these peptides had an antioxidant effect of removing active oxygen species, peroxides and the like from living organisms by promoting glutathione synthetase gene expression, and hence synthesis of glutathione.

### Example 2

### [Preparation of Sample]

### <Preparation of Collagen Peptide Mixture>

As a collagen peptide mixture comprising both or one of the peptides of Gly-Pro (GP) and Glu-Hyp-Gly (BOG), a collagen peptide mixture D (trade name:

“COLLAGENAID”, manufactured by Nitta Gelatin Inc., weight average molecular weight (Mw): about 4,000 Da) was prepared. The collagen peptide mixture D contained “EOG” and “GP” at a total of 132 ppm in quantitative analysis performed by LC-MS/MS under the same conditions as in [Example 1] described above.

[Aging Progression Suppression Test on Humans]

The collagen peptide mixture D was administered to a total of 95 subjects in their 10s to 70s (2 males and 92 females), and whether or not the subjects sensed an aging progression suppressive effect was examined. Specifically, the collagen peptide mixture D was orally administered to the 95 subjects for 10 to 20 days (14 days on average) at 4 to 6 g a day without specifying the administration time. Thereafter, subjects who had sensed an aging progression suppressive effect were interviewed about (surveyed on) the relevant parts and details of the effect (specific contents).

Tables 5 to 10 show the results. Table 5 shows the parts at which the aging progression suppressive effect was sensed, and the number of subjects who sensed the aging progression suppressive effect at each of the parts (multiple answers allowed). Table 6 shows specific contents when the aging progression suppressive effect was sensed at the skin, and the number of subjects who gave such contents (multiple answers allowed). Table 7 shows specific contents when the aging progression suppressive effect was sensed in the hair, and the number of subjects who gave such contents (multiple answers allowed). Table 8 shows specific contents when the aging progression suppressive effect was sensed in the nail, and the number of subjects who gave such contents (multiple answers allowed). Table 9 shows specific contents when the aging progression suppressive effect was sensed in the joint, and the number of subjects who gave such contents (multiple answers allowed). Table 10 shows specific contents when the aging progression suppressive effect was sensed in other parts, and the number of subjects who gave such contents (multiple answers allowed).

TABLE 5

| Part at which effect is sensed | Number of subjects |
|---|---|
| Skin | 46 |
| Joint | 6 |
| Bone | 1 |
| Nail | 22 |
| Hair | 24 |
| Other parts | 5 |

TABLE 6

| Skin: details (contents) of effect | Number of subjects |
|---|---|
| Improvement in elasticity | 8 |
| Improvement in sagging | 2 |
| Improvement in dryness/moistness | 15 |
| Improvement in cuticle roughness | 2 |
| Improvement in texture | 2 |
| Improvement in smoothness of makeup | 3 |
| Improvement in wrinkles | 4 |
| Improvement in follicles | 1 |
| Improvement in chapped hand | 3 |
| Improvement in skin brightness | 2 |
| Elasticity | 2 |
| Gloss | 5 |
| Fluffy/springy feeling | 3 |
| Smoothness/smooth feeling | 3 |
| Firmness of entire face | 1 |
| Decrease in pimples | 1 |
| Improvement in blotches | 1 |
| Total | 58 |

TABLE 7

| Hair: details (contents) of effect | Number of subjects |
|---|---|
| Improvement in gloss | 4 |
| Improvement in dryness (looseness) | 4 |
| Decrease in hair loss | 4 |
| Improvement in settlement | 2 |
| Improvement in combability | 2 |
| Dry feeling | 2 |
| Improvement in hair thickness | 2 |
| Decrease in gray hair | 1 |
| Improvement in softness | 1 |
| Improvement in hair stiffness | 1 |
| Increase in volume of hair | 1 |
| growth of hair | 1 |
| Total | 25 |

TABLE 8

| Nail: details (contents) of effect | Number of subjects |
|---|---|
| Improvement in fragility | 3 |
| Gloss | 1 |
| Toughness | 1 |
| Total | 5 |

TABLE 9

| Joint: details (contents) of effect | Number of subjects |
|---|---|
| Improvement in joint pain | 3 |
| Joint sounding | 1 |
| Improvement in feeling of strangeness | 1 |
| Total | 5 |

TABLE 10

| Others: details (contents) of effect | Number of subjects |
|---|---|
| Improvement in bowel movement | 3 |
| Elasticity of breast | 1 |
| Total | 4 |

[Discussions]

From Tables 5 to 10, it is understood that the collagen peptide mixture D (aging progression suppressing agent) comprising both or one of the peptides of Gly-Pro (GP) and Glu-Hyp-Gly (EOG) exhibits an aging progression suppressive effect in the skin, the hair, the nail, the joint and other parts.

While embodiments and Examples of the present invention have been described above, the configurations of the embodiments and Examples described above may be appropriately combined as originally envisioned.

The embodiments and Examples disclosed herein should be regarded as illustrative rather than limiting in any way. The scope of the present invention is given by the appended claims rather than the foregoing description, and all changes which fall within the range of the appended claims and equivalents thereof are intended to be embraced therein.

1. An aging progression suppressing agent comprising both or one of the peptides of Gly-Pro and Glu-Hyp-Gly, a salt thereof, or a chemically modified product thereof.

2. The aging progression suppressing agent according to claim 1, wherein the peptides are derived from collagen.

3. The aging progression suppressing agent according to claim 1, wherein the aging progression suppressing agent is a collagen peptide mixture.

4. The aging progression suppressing agent according to claim 3, wherein the collagen peptide mixture has a weight average molecular weight of 100 Da or more and 5,000 Da or less.

5. The aging progression suppressing agent according to claim 1, wherein the aging progression suppressing agent is a promoter of type 17 collagen gene expression or a promoter of glutathione synthetase gene expression.

6. A food or beverage product comprising the aging progression suppressing agent according to claim 1.
